(19) United States
(12) Patent Application Publication
(10) Pub. No.: US 2005/0142209 A1
(43) Pub. Date: Jun. 30, 2005

(54) Aqueous alkaline solution for mineral supplementation, preparing method thereof and composition for prevention and improvement of osteoporosis containing the same

(76) Inventors: Seong-Yung Hwa, Jinju-Si (KR); Young-Chul Jung, Jinju-Si (KR); Sung-Sik Chun, Jinju-Si (KR); Shin-Kwon Kang, Jinju-Si (KR); Jong-Kwon Seo, Jinju-Si (KR)

Correspondence Address: PARK & SUTTON LLP, 3255 WILSHIRE BLVD, SUITE 1110, LOS ANGELES, CA, 90010, US

(21) Appl. No.: 10/823,915

(22) Filed: Apr. 14, 2004

(30) Foreign Application Priority Data
Apr. 14, 2003 (KR) 10-2003-0023431

(51) Int. Cl. A61K 35/78; A61K 35/80; A61K 38/17; A61K 35/32; A61K 31/19; A61K 31/202

(52) U.S. Cl. 424/549; 424/195.17; 424/757; 514/7; 514/560; 514/574; 514/557; 424/764; 424/773

(57) ABSTRACT

The present invention relates to an aqueous alkaline solution for mineral supplementation comprising bovine bones, cuttlefish bones, red algae and an organic acid. Also, the invention relates to a method for preparing the aqueous alkaline solution, and a composition or health food containing the aqueous alkaline solution, which is effective in the prevention and improvement in osteoporosis. The aqueous alkaline solution contains various minerals necessary for the human body at large amounts, and thus, can be used as a mineral supplement. Also, the composition prevents bone absorption and bone mineral absorption, and thus, is useful for the prevention and improvement of bone diseases, such as osteoporosis and degenerative bone diseases.

### CLAIMING FOREIGN PRIORITY

The applicant claims and requests a foreign priority, through the Paris Convention for the Protection of Industry Property, based on a patent application filed in the Republic of Korea (South Korea) with the filing date of Apr. 14, 2003, with the application number 10-2003-0023431, by the applicant. (See the Attached Declaration).

### BACKGROUND OF THE INVENTION

1. Field of the Invention

The present invention relates to an alkaline solution for mineral supplementation, a preparing method thereof, and a composition for the prevention and improvement of osteoporosis containing the same. More particularly, the present invention relates to an alkaline solution for mineral supplementation containing bovine bones, cuttlefish bones, red algae and organic acids, a preparing method thereof, and a composition or food containing the same, which is effective in the prevention and improvement of osteoporosis.

2. Background of the Related Art

Minerals activate the metabolism of carbohydrate, fat and protein to promote the physiological activity and growth of a living body, thus acting to activate all nutrients. The human body needs relatively large amounts (i.e., more than 100 mg per day) of the minerals, such as calcium (Ca), phosphorus (P), sodium (Na), potassium (K), chlorine (Cl), magnesium (Mg) and sulfur (S). Furthermore, the human body needs a very small amount of other minerals, such as iron (Fe), cobalt (Co), zinc (Zn), manganese (Mn), iodine (I), molybdenum (Mn), selenium (Se), fluorine (F) and chromium (Cr). Although the minerals are present in the human body at trace amounts, they play important roles. For example, they help to maintain the energy, growth and tissue of the human body, and regulate the body activity. However, lack of the minerals causes problems in body tissue and metabolism progression.

The bioavailability of the minerals varies depending on the following factors: the intake level and chemical form of micronutrients, the presence of substances interfering with the absorption of the micronutrients, and the interaction between the micronutrients and other nutrient. For this reason, researches on suitable intake and deficiency of the micronutrients are being actively conducted.

Factors influencing the bioavilability of the minerals are broadly divided into endogenous factors and exogenous factors. The endogenous factors include age, sex, health and disease conditions, and pregnancy, and the exogenous factors include the ingestion of protein, fat, carbohydrate and vitamin. Particularly, the ingestion of a suitable level of the minerals are necessary for modern persons who live largely on cereals or often take convenience foods made of refined cereals.

Of minerals playing an important role in the mechanism of the human body's metabolism, calcium is involved in the calcification of bones and the coagulation of blood, and magnesium acts to inhibit the excitement of muscles and nerves. Also, iron is a component of hemoglobin and acts as a coenzyme of various enzymes, and zinc is a coenzyme of RNA polymerase. Copper is a coenzyme of superoxide dismustase, and cobalt is a component of vitamin B₁₂ playing an important role in the prevention and improvement of traumatic anemia. Thus, such minerals play an important role in the development of various diseases.

As the industrial industry is developed and the intake of convenience food is increased, calcium requirement in modern persons is increased, and thus, calcium deficiency frequently occurs. This is because the actual absorption rate of calcium is low, thus causing a problem in view of calcium availability, although the intake of calcium was increased. In an attempt to solve this problem, various calcium salts of different chemical forms, calcium-enriched foods based on eggshells or oyster shells, calcium supplements, and substances for increasing calcium availability in vivo, are being recently developed in various countries. Also, the effectiveness and nutritional effect of such calcium sources are being examined by animal tests in various manners.

Osteoporosis is a condition where calcium in bone tissue is reduced so that the compact substance of bones is lost, thus widening the medullary cavity. As this condition is progressed, bones become weak and are liable to fracture even at low shock pressure. Bone mass is influenced by various factors, such as genetic factors, nutrient intake, hormone change, and differences in exercises and life habits, and osteoporosis is known to be caused by old age, insufficient exercise, low body weight, smoking, low-calcium diet, menopause, ovariectomy and the like. Meanwhile, although there is a difference in bone mass between individuals, Negroes have a higher bone mass than that of white men due to their low bone resorption level. The bone mass is the highest for persons aged 14-18 years, and reduced by 1% a year in the winter of life. Particularly in woven, from 30-years old, the bone mass is continuously decreased, and when reaching the menopause, the bone mass is rapidly decreased due to hormone change.

As described above, osteoporosis is an inevitable condition in old-aged persons, particularly postmenopausal women, although there is a difference in the degree of osteoporosis. With an aging population trend in highly advanced countries, interests in osteoporosis and agents for treating the same are gradually increased. With respect to the treatment of bone diseases, a worldwide market of about 1,300 US billion dollars is formed and expected to further increase in future. For this reason, worldwide research institutes and pharmaceutical companies are making significant investment in the development of agents for treating the bone diseases.

### SUMMARY OF THE INVENTION

Accordingly, an object of the present invention is to provide an aqueous alkaline solution for mineral supplementation containing large amounts of minerals necessary for the human body, such as calcium, phosphorus, sodium, potassium and zinc, as well as a preparing method thereof.

Another object of the present invention is to provide a composition and health food effective in the prevention and improvement of osteoporosis.

To achieve the above objects, in one aspect, the present invention an aqueous alkaline solution for mineral supplementation, which comprises bovine bones, cuttlefish bones, red algae, an organic acid and purified water.

In another aspect, the present invention provides a composition for the prevention and improvement of osteoporosis, which comprises: (a) the aqueous alkaline solution for mineral supplementation according to the present invention; (b) a medicinal plant; (c) a yellow dried Alaska pollack; (d) black beans; (d) Lentinus edodes, (f) casein phospeptide; and (g) docosahexaenoic acid (DHA).

In still another aspect, the present invention provides a preparing method of an aqueous alkaline solution for mineral supplementation, the method comprising the steps of: (a) pulverizing bovine bones, cuttlefish bones and red algae to make powders; (b) heating the powders at a temperature of 1,000-1,200° C.; (c) cooling the heated powders; (d) adding purified water to the cooled powders to make a solution; (e) adding an organic acid to the solution; (f) solubilizing the organic acid-containing solution in a pressurized extractor at a temperature of 120-150° C., and (g) cooling and filtering the solubilized solution.

### DETAILED DESCRIPTION OF THE PREFERRED EMBODIMENT

Hereinafter, the present invention will be described in detail.

During our researches to develop a solution for mineral supplementation and an agent for the prevention and improvement of osteoporosis, the present inventors have prepared an aqueous solution by adding an organic acid and purified water to minerals obtained by calcining bovine bones, cuttlefish bones and red algae, and consequently, found that the aqueous solution contains various minerals at large amounts, and a composition containing the aqueous solution has positive effects on the prevention and improvement of osteoporosis. On the basis of this discovery, the present invention was completed.

An aqueous alkaline solution for mineral supplementation according to the present invention comprises bovine bones, cuttlefish bones, red algae, organic acids, and purified water. The bovine bones, the cuttlefish bones and the red algae can be used in the form of a powder obtained by high-temperature calcination. The red algae belong to marine algae together with green algae and brown algae, and examples of the red algae include layer and agar-agar. The content of the bovine bones, the cuttlefish bones and the red algae is preferably 2-10% by weight relative to the weight of the aqueous solution. If the content of the bovine bones, the cuttlefish bones and the red algae is less than 2% by weight, the mineral supplementation effect of the resulting aqueous solution will be insufficient. If the content is more than 10% by weight, the dissolution of the inorganic substances will reach a saturation state so that their solubility will not be increased.

The organic acid, which is used in the present invention, is preferably at least one selected from the group consisting of acetic acid, lactic acid and citric acid. The content of the organic acid is preferably 2-10% by weight relative to the weight of the aqueous solution. If the organic acid content is less than 2% by weight, the solubility of the minerals will not be increased, and if it is more than 10%, the resulting aqueous solution will have high acidity.

Hereinafter, a preparing method of the aqueous alkaline solution for mineral supplementation will be described in detail.

In the first step, bovine bones, cuttlefish bones and red algae are pulverized to form fine powders.

In the second step, the powders are calcined by heating at a temperature of 1,000-1,200° C. By this heating step, various bacteria and impurities are completely combusted and removed, leaving only minerals. This heating step is preferably performed for 30 minutes to one hour. If the heating time is shorter than 30 minutes, the degree of calcination of the minerals will be lowered, and if the heating time is longer than one hour, the degree of calcination of the minerals will not be further increased.

In the third step, the heated powders are cooled at room temperature and then added with purified water to make a solution. The content of the powders is preferably 2-10% by weight relative to the weight of the aqueous solution. If the content of the powders is less than 2% by weight, the mineral supplementation effect of the resulting solution will be insignificant, and if the content is more than 10% by weight, the dissolution of the minerals will reach a saturation state so that their solubility will not be increased.

In the fourth step, an organic acid is added to the prepared solution. The organic acid is preferably at least one selected from the group consisting of acetic acid, lactic acid and citric acid. The content of the organic acid is preferably 2-10% by weight relative to the weight of the aqueous solution. If the content of the organic acid is less than 2% by weight, the dissolution of the minerals will be insufficient, and if the content is more than 10% by weight, the solubility of the minerals will not be increased and also the acidity of the resulting aqueous solution will be increased.

In the fifth step, the organic acid-containing solution is solubilized in a pressurized extractor at a temperature of 120-150° C. If the solubilization step is preferably conducted for 20 minutes to one hour. If the solubilization time is shorter than 20 minutes, the solubilization will be insufficient, and if it is longer than one hour, the solubility of the minerals will not be increased.

In the sixth step, the solubilized solution is cooled and filtered.

The aqueous alkaline solution for mineral supplementation according to the present invention is characterized by containing bovine bones, cuttlefish bones, red algae and an organic acid. In addition to such components, the aqueous alkaline solution of the present invention may also contain sweeteners or acidulants. The content of such additives is not specifically limited and can be easily selected by a person skilled in the art.

The present invention provides a composition for the prevention and improvement of osteoporosis, which comprises: the aqueous alkaline solution for mineral supplementation according to the present invention or the aqueous solution for mineral supplementation prepared by the inventive preparing method; a medicinal plant; a yellow dried Alaska pollack; black beans; Lentinus edodes; casein phospeptide; and docosahexaenoic acid (DHA).

The medicinal plant is selected from Eucommiae Cortex, Cervi cornu, Dioscoreae Rhizoma, Crataegi Fructus, Poria cocos, steamed Rehmannia glutinosa, Acori Graminei Rhizoma, Acori Graminei Rhizoma, Astragali Radix, Paeonia lactiflora Pallas, Cnidii Rhizoma, Angelicae Giganis Radiz, Glycyrrhiza and a mixture thereof. The content of the medicinal plant is preferably 0.1-27.5% by weight relative to the weight of the composition. If the content of the medicine plant is more than 27.5% by weight, it will be difficult to formulate the composition into a form, such as a pill.

The content of the yellow dried Alaska pollack is preferably 0.1-20% by weight relative to the weight of the composition. If the content of the yellow dried Alaska pollack is more than 20% by weight, it will not be easy to formulate the composition into a form, such as a pill.

The content of the black beans is preferably 0.1-20% by weight relative to the weight of the composition. If the content of the black beans is more than 20% by weight, it will not be easy to formulate the composition into a form, such as a pill.

The content of the Lentinus edodes is preferably 0.1-10% by weight relative to the weight of the composition. If the content of the Lentinus edodes is more than 10% by weight, it will not be easy to formulate the composition into a form, such as a pill.

The content of the casein phospeptide is preferably 0.1-1.2% by weight relative to the weight of the composition. If the content of the casein phospeptide is more than 1.2% by weight, an effect caused by addition of the casein phospeptide will no longer increase.

The content of the docosahexaenoic acid (DHA) is preferably 0.1-0.3% by weight relative to the weight of the composition. If the DHA content is more than 0.3% by weight, an effect caused by the DHA addition will not be further increased.

Furthermore, the present invention provides a health food, which contains the composition for the prevention and improvement of osteoporosis according to the present invention, as an active ingredient. Examples of the health food, which contains the composition for the prevention and improvement of osteoporosis according to the present invention, as an active ingredient, include health and favorite foods, such as juice, tea and jelly. The formulation of the inventive health food is preferably a pill.

The present invention will hereinafter be described in further detail by examples. It will however be obvious to a person skilled in the art that that the present invention is not limited to or by the examples.

### EXAMPLE 1

1. 1 kg of washed bovine bones, 1 kg of cuttlefish bones and 1 kg of red algae are ground into fine powders.

2. The powders are calcined by heating at 1,000-1,200° C. for 30 minutes to obtain minerals.

3. The heated powders are cooled at room temperature.

4. 1 liter of purified water was added to 50 g of the cooled powder to make a solution.

5.50 g of acetic acid is added to the solution, and stirred slowly for 2 hours under reduced pressure while solubilizing the minerals.

6. The organic acid-containing solution is solubilized in a pressurized extractor at 130° C. for 30 minutes.

7. The solubilized solution is cooled at room temperature and filtered through a filter paper to give an aqueous alkaline solution for mineral supplementation.

### EXAMPLE 2

1.5 g of each of Eucommiae Cortex, Cervi cornu, Dioscoreae Rhizoma, Crataegi Fructus, Poria cocos, steamed Rehmannia glutinosa, Acori Graminei Rhizoma, Astragali Radix, Paeonia lactiflora Pallas, Cnidii Rhizoma, Angelicae Giganis Radiz, and Glycyrrhiza are added to 1 kg of the aqueous alkaline solution for mineral supplementation prepared in Example 1. The mixture is extracted at 100° C. for 5 hours, filtered, concentrated under reduced pressure, and adjusted to a concentration of 20-25 Brix.

2. Then, the resulting material is added with 20 g of a yellow dried Alaska pollack, 20 g of black bean powders, 10 g of Lentinus edodes powders, 12 g of casein phospeptide, and 3 g of DHA, to prepare a composition.

### EXAMPLE 3

1. The composition prepared in Example 2 is mixed and kneaded. The kneaded material is formulated into a pill using a pill-making machine.

2. The formulated pill is dried in a drier at 40-50° C. to a water content of less than 8%.

### TEST EXAMPLE 1

According to a trace element analysis method described in Korean food code, content analysis for nine minerals, including calcium, was performed on the aqueous alkaline solution for mineral supplementation prepared in Example 1.

The content analysis results for the aqueous alkaline solution for mineral supplementation are given in Table 1 below.

TABLE 1

| Minerals | Content (mg/100 g) |
|---|---|
| Calcium | 1,300 |
| Magnesium | 125 |
| Phosphorus | 107 |
| Sodium | 1,002 |
| Potassium | 285 |
| Zinc | 86 |
| Copper | 75 |
| Manganese | 29 |
| Cobalt | 18 |

From Table 1 above, it can be found that the aqueous alkaline solution for mineral supplementation according to the present invention contains various minerals at large amounts.

### TEST EXAMPLE 2

In order to examine if the composition according to the present invention is effective in the prevention and improvement of osteoporosis, the following test was performed using the composition prepared in Example 2.

1. Test Method

(1) Test Material

The composition prepared in Example 2 was used in the following test.

Spraque-Dawley female white rate were adapted to the test environment for about 3 weeks while supplying with a sufficient amount of solid feed and water. Of such rats, the rats weighing about 200 g were divided into three groups: a normal group (non-ovariectomized), a control group (ovariectomized, and administered with basic feed), and a test group (ovariectomized, and administered with basic feed and the inventive composition). Each group consists of 10 animals.

Ovariectomy was performed as follows.

1 ml/kg body weight of ketamine was administered into the abdominal cavity of the white rats to anesthetize the rats. After anesthetizing the rats, hair on the back side of the white rats was removed with an electric razor, and the back side from which the hair had been removed was sterilized with 70% alcohol. Then, about 3 cm of skin tissue along the spinal line below the back side of the white rats was incised with a scalpel, and both sides of the peritoneum where the ovary is located were incised to a length of 1.5 cm, after which the ovary was removed. In the control and test groups, the left ovary was first removed, and the normal group was sutured without removing the ovary. At one day after the surgical operation, the rats were with administered with an antibiotic agent, and then subjected to a recovery stage of about four weeks.

(3) Administration of Feed Compositions

The normal group, the control group and the test group were administered with compositions given in Table 2 below, respectively.

Daily feed amount for the white rats used in the clinical test was 60 g/kg body weight, and daily feed intake for the white rats was the average of feed intakes that the white rats take for 3 days. The feed compositions were administered for 60 days and then analyzed for their osteoporosis relief effect.

TABLE 2

| Groups | Feed compositions |
|---|---|
| Normal group | 100% basic feed |
| (non-ovariectomized) | |
| Test group | 60 wt % basic feed + 40 wt % composition |
| (ovariectomized) | prepared in Example 2 |
| Control group | 100% basic feed |
| (ovariectomized) | |

(4) Change in Serum Components

(a) Blood Collection and Serum Separation

At 60 days after surgical removal of the ovary, 1.0 ml of ketamine hydrochloride (commercially available of the trade name of KETARA from Yuhan corporation, Korea) was injected into the abdominal cavity of each white rat to anesthetize the rat. Then, blood was collected from the heart, and left to stand at room temperature for 30 minutes. Then, the blood was centrifuged at 3,000 rpm for 15 minutes to separate serum.

(b) Measurement of Serum Osteocalcine Level

For the measurement of serum osteocalcine level, ELSA-OSETO kit (CIS biointernational, France) as a regent and ICN biomedicals (ISOMEDIC 10/600, USA) as a device were used.

(c) Measurement of Serum Calcium Level

For the measurement of serum calcium level, calcium-HRII kit (Wako pure chemical industries, Ltd., Japan) as a regent and Hitachi 747 (Automatic chemistry analyzer, Japan) as a device were used.

(d) Measurement of Serum Phosphatase (ALP) Activity

For the measurement of serum alkaline phosphatase activity, a regent for automatic ALP measurement (Asan Phamaceutical Co, Ltd., Korea) and Hitachi 747 (Automatic chemistry analyzer, Japan) as a device were used.

(e) Measurement of Serum Phosphorus Activity

For the measurement of serum phosphorus activity, a regent for automatic phosphorus measurement (Asan Phamaceutical Co, Ltd., Korea) and Hitachi 747 (Automatic chemistry analyzer, Japan) as a device were used.

(5) Change in Urine Components

(a) Urine Collection and Measurement of Urine Amount

At 59 days after surgical removal of the ovary, the white rats were individually placed into a plastic cage for white rat metabolism while allowing free access to water and feed. Urine was collected from the white rat for 24 hours, weighed and centrifuged at 3,000 rpm for 15 minutes to collect the supernatant urine.

(b) Measurement of Urine Creatinine Level

For the measurement of urine creatinine level, Creatin kit (Daiichi, Japan) as a reagent and Hitachi 747 (Automatic chemical analyzer, Japan) as a device were used.

(c) Measurement of Urine Calcium Level

For the measurement of urine calcium level, calcium-HRII kit (Wako pure chemical industries, Ltd., Japan) as a reagent and Hitachi 747 (Automatic chemistry analyzer, Japan) as a device were used.

(d) Measurement of Urine Pyridinoline Level

For the measurement of urine pyridinoline level, Pyrilinks-G kit (Metra biosystem, USA) as a reagent and Pasteur ELISA system (LP400 LP35) as a device were used.

2. Test Results

(1) Change in Serum Components

When the ovary of rats is experimentally removed, the number of osteoblasts and osteoclasts in trabecular bones will be increased and the activity of the osteoclasts will be superior to that of the osteoblasts, so that the amount of bone mass will be reduced. However, the administration of female hormone estrogen inhibits an increase in the osteoblast and osteoclast number. Bone mass decrease after ovariectomy results in an increase in serum osteocalcine, calcium and alkaline phosphatase levels, etc., which are used as an index to evaluate bone turnover rate.

In the present invention, osteoporosis by estrogen deficiency was induced in the adult white rats by ovariectomy. Then, the composition prepared in Example 2 was administered to the white rats for 60 days, after which serum osteocalcine, calcium, alkaline phosphatase (ALP) and phosphorus levels were measured.

The measurement results showed that the serum osteocalcine level, which is an index to evaluate bone mineral metabolism, was far higher in the test group administered with the composition of Example 2 than that in the control group. This suggests that the inventive composition is effective in inhibiting a change in bone turnover rate caused by ovariectomy.

The serum calcium and phosphorus levels were not significantly different between the normal group, the control group and the test group.

The serum ALP level, which is used as a useful index to evaluate osteogenetic activity in patients with bone diseases, was significantly higher in the test group than that in the other group. This suggests that the inventive composition has osteogenetic effect.

Table 3 below shows a change in the serum components of the ovariectomized white rats.

TABLE 3

| Serum components | Normal group | Control group | Test group |
|---|---|---|---|
| Osteocalcine (mg/l) | 0.21 | 0.09 | 0.18 |
| Calcium (mg/dl) | 10.5 | 10.4 | 10.5 |
| APL (IU/l) | 192 | 283 | 410 |
| Phosphorus (mg/dl) | 7.2 | 7.3 | 7.4 |

(2) Change in Urine Components

Table 4 below shows a change in urine components of the ovariectomized white rats.

TABLE 4

| Urine components | Normal group | Control group | Test group |
|---|---|---|---|
| Urine amount | 3.6 | 10.2 | 3.9 |
| Deoxypyridinoline/creatine | 20.3 | 17.6 | 10.5 |
| (nM/mM) | | | |
| Calcium/creatine (mg/g) | 32.4 | 68.5 | 29.8 |

The amount of urine discharged for 24 hours in the test group was similar to that in the normal group, but significantly lower than that in the control group. This suggests that the inventive composition has a positive effect on the improvement of osteoporosis.

Deoxypyridinoline is found mainly in bones, and its level in urine is used as an index to evaluate bone absorption. The deoxypyridinoline/creatine ratio was the lowest for the test group, and the calcium/creatine ratio was the lowest for the control group. Such results indicate that the inventive composition used in this test is effective in preventing bone absorption and bone mineral loss so that it reduces urine deoxypyridinoline and calcium levels.

As described above, the aqueous alkaline solution for mineral supplementation according to the present invention contains various minerals necessary for the human body at large amounts, and thus, can be used as a mineral supplement.

Furthermore, the composition for the prevention and improvement of osteoporosis according to the present invention prevents bone absorption and bone mineral absorption, and thus, is useful for the prevention and improvement of bone diseases, such as osteoporosis and degenerative bone diseases. In addition, the inventive composition is non-toxic, and thus, can be widely used as health foods.

1. An aqueous alkaline solution for mineral supplementation, which comprises bovine bones, cuttlefish bones, red algae, an organic acid and purified water, wherein:
(a) the organic acid is at least one selected from the group consisting of acetic acid, lactic acid and citric acid;
(b) the content of the bovine bones, the cuttlefish bones and the red algae is 2-10% by weight relative to the weight of the aqueous solution; and
(c) the content of the organic acid is 2-10% by weight relative to the weight of the aqueous solution.

2. A composition for the prevention and improvement of osteoporosis, which comprises:
(a) the aqueous alkaline solution for mineral supplementation as set forth in claim 1;
(b) a medicinal plant;
(c) a yellow dried Alaska pollack;
(d) black beans;
(e) Lentinus edodes;
(f) casein phospeptide; and
(g) docosahexaenoic acid (DHA).

3. The composition of claim 2, wherein the medicinal plant is selected from the group consisting of Eucommiae Cortex, Cervi cornu, Dioscoreae Rhizoma, Crataegi Fructus, Poria cocos, steamed Rehmannia glutinosa, Acori Graminei Rhizoma, Astragali Radix, Paeonia lactiflora Pallas, Cnidii Rhizoma, Angelicae Giganis Radiz, Glycyrrhiza and a mixture thereof.

4. The composition of claim 2, wherein the content of the yellow dried Alaska pollack is 0.1-20% by weight relative to the weight of the composition.

5. The composition of claim 2, wherein the content of the black beans is 0.1-20% by weight relative to the weight of the composition.

6. The composition of claim 2, wherein the content of the medicinal plant is 0.1-27.5% by weight relative to the weight of the composition.

7. The composition of claim 2, wherein the content of the Lentinus edodes is 0.1-10% by weight relative to the weight of the composition.

8. The composition of claim 2, wherein the content of the casein phospeptide is 0.1-1.2% by weight relative to the weight of the composition.

9. The composition of claim 2, wherein the content of the docosahexaenoic acid (DHA) is 0.1-0.3% by weight relative to the weight of the composition.

10. A health food, which comprises the composition for the prevention and improvement of osteoporosis as set forth in claim 2, as an active ingredient, wherein the formulation of the health food is a pill.

11. The health food of claim 10, wherein the medicinal plant is selected from the group consisting of Eucommiae Cortex, Cervi cornu, Dioscoreae Rhizoma, Crataegi Fructus, Poria cocos, steamed Rehmannia glutinosa, Acori Graminei Rhizoma, Astragali Radix, Paeonia lactiflora Pallas, Cnidii Rhizoma, Angelicae Giganis Radiz, Glycyrrhiza and a mixture thereof.

12. The health food of claim 10, wherein the content of the yellow dried Alaska pollack is 0.1-20% by weight relative to the weight of the composition.

13. The health food of claim 10, wherein the content of the black beans is 0.1-20% by weight relative to the weight of the composition.

14. The health food of claim 10, wherein the content of the medicinal plant is 0.1-27.5% by weight relative to the weight of the composition.

15. The health food of claim 10, wherein the content of the Lentinus edodes is 0.1-10% by weight relative to the weight of the composition.

16. The health food of claim 10, wherein the content of the casein phospeptide is 0.1-1.2% by weight relative to the weight of the composition.

17. The health food of claim 10, wherein the content of the docosahexaenoic acid (DHA) is 0.1-0.3% by weight relative to the weight of the composition.

18. A preparing method of an aqueous alkaline solution for mineral supplementation, which comprises the steps of:
(a) pulverizing bovine bones, cuttlefish bones and red algae to make powders;
(b) heating the powders at a temperature of 1,000-1,200° C. for 30 minutes to one hour;
(c) cooling the heated powders;
(d) adding purified water to the cooled powders to make a solution, wherein the content of the powders is 2-10% by weight relative to the weight of the aqueous solution;
(e) adding an organic acid to the solution, wherein the content of the organic acid is 2-10% by weight relative to the weight of the aqueous solution;
(f) solubilizing the organic acid-containing solution in a pressurized extractor at a temperature of 120-150° C. for 20 minutes to one hour; and
(g) cooling and filtering the solubilized solution, wherein: the organic acid is at least one selected from the group consisting of acetic acid, lactic acid and citric acid.

19. A composition for the prevention and improvement of osteoporosis, which comprises:
(a) the aqueous alkaline solution for mineral supplementation prepared by the method of claim 18;
(b) a medicinal plant;
(c) a yellow dried Alaska pollack;
(d) black beans;
(e) Lentinus edodes;
(f) casein phospeptide; and
(g) docosahexaenoic acid (DHA).

20. The composition of claim 19, wherein the medicinal plant is selected from the group consisting of Eucommiae Cortex, Cervi cornu, Dioscoreae Rhizoma, Crataegi Fructus, Poria cocos, steamed Rehmannia glutinosa, Acori Graminei Rhizoma, Astragali Radix, Paeonia lactiflora Pallas, Cnidii Rhizoma, Angelicae Giganis Radiz, Glycyrrhiza and a mixture thereof.

21. The composition of claim 19, wherein the content of the yellow dried Alaska pollack is 0.1-20% by weight relative to the weight of the composition.

22. The composition of claim 19, wherein the content of the black beans is 0.1-20% by weight relative to the weight of the composition.

23. The composition of claim 19, wherein the content of the medicinal plant is 0.1-27.5% by weight relative to the weight of the composition.

24. The composition of claim 19, wherein the content of the Lentinus edodes is 0.1-10% by weight relative to the weight of the composition.

25. The composition of claim 19, wherein the content of the casein phospeptide is 0.1-1.2% by weight relative to the weight of the composition.

26. The composition of claim 19, wherein the content of the docosahexaenoic acid (DHA) is 0.1-0.3% by weight relative to the weight of the composition.

27. A health food, which comprises the composition for the prevention and improvement of osteoporosis as set forth in claim 19, as an active ingredient, wherein the formulation of the health food is a pill.

28. The health food of claim 27, wherein the medicinal plant is selected from the group consisting of Eucommiae Cortex, Cervi cornu, Dioscoreae Rhizoma, Crataegi Fructus, Poria cocos, steamed Rehmannia glutinosa, Acori Graminei Rhizoma, Astragali Radix, Paeonia lactiflora Pallas, Cnidii Rhizoma, Angelicae Giganis Radiz, Glycyrrhiza and a mixture thereof.

29. The health food of claim 27, wherein the content of the yellow dried Alaska pollack is 0.1-20% by weight relative to the weight of the composition.

30. The health food of claim 27, wherein the content of the black beans is 0.1-20% by weight relative to the weight of the composition.

31. The health food of claim 27, wherein the content of the medicinal plant is 0.1-27.5% by weight relative to the weight of the composition.

32. The health food of claim 27, wherein the content of the Lentinus edodes is 0.1-10% by weight relative to the weight of the composition.

33. The health food of claim 27, wherein the content of the casein phospeptide is 0.1-1.2% by weight relative to the weight of the composition.

34. The health food of claim 27, wherein the content of the docosahexaenoic acid (DHA) is 0.1-0.3% by weight relative to the weight of the composition.
